(19) United States
(12) Patent Application Publication
(10) Pub. No.: US 2002/0173762 A1
(43) Pub. Date: Nov. 21, 2002

(54) Body fluid absorbent article

(76) Inventors: Norihiko Ishikawa, Kagawa-ken (JP); Toshio Inoue, Kagawa-ken (JP); Kozo Abe, Kagawa-ken (JP)

Correspondence Address: BAKER & DANIELS, 111 E. WAYNE STREET, SUITE 800, FORT WAYNE, IN, 46802

(21) Appl. No.: 10/139,948

(22) Filed: May 6, 2002

(30) Foreign Application Priority Data
May 7, 2001 (JP) 2001-136404

(51) Int. Cl. A61F 13/15; A61F 13/20

(52) U.S. Cl. 604/385.01

(57) ABSTRACT

A body fluid absorbent article includes an absorbent material, which includes a woven fabric or a knitted fabric made of yarns which are composed of super-absorbent polymer and synthetic fibers and/or regenerated fibers united.

### BACKGROUND OF THE INVENTION

This invention relates to body fluid absorbent articles such as disposable diapers, menstruation pads and the like for absorption and containment of body fluids.

Japanese Patent Application No. 1996-196559A discloses a disposable diaper having a liquid-absorbent core disposed between a liquid-pervious topsheet and a liquid-impervious backsheet wherein the liquid-absorbent core comprises a fibrous assembly layer placed upon a liquid-retaining layer formed by a mixture of fluff pulp and super-absorbent polymer particles.

The liquid-retaining layer and the fibrous assembly layer of the core are intermittently joined together substantially over the entire surfaces thereof opposed to each other and these two layers are integrally covered with tissue paper. These two layers and the tissue paper integrally covering them are also intermittently joined together substantially over the entire surfaces thereof opposed to each other. This diaper is claimed to be able to maintain the liquid-retaining layer, the fibrous assembly layer and the tissue paper in close contact with one another even when the diaper is bent or twisted. It is also claimed that the liquid-retaining layer is prevented from losing its shape even though this layer is less flexible and apt to get loose due to a large quantity of super-absorbent polymer particles contained therein.

With the diaper disclosed in the above-cited Publication, if the tissue paper is torn during use of the diaper, the liquid-retaining layer and the tissue paper may be peeled off from each other and the diaper may not keep its original shape. Once having been out of shape, the liquid-retaining layer can not restore its initial shape. In order that the core may achieve its proper function, the core is preferably formed by the liquid-retaining layer alone. However, in the case of the diaper disclosed by the above-cited Publication, the liquid-retaining layer itself has no means adapted to prevent this layer from losing its original shape.

### SUMMARY OF THE INVENTION

In an aspect of the invention, a body fluid absorbent article comprises:

an absorbent material comprising at least one of a woven fabric and a knitted fabric made of yarns which are composed of super-absorbent polymer particles and at least one of synthetic fibers and regenerated fibers which are united with said absorbent polymer particles.

In another aspect of the invention, a body fluid absorbent article comprises:

an absorbent material comprising at least one of a woven fabric and a knitted fabric made of yarns which are composed of super-absorbent fibers obtained by spinning a super-absorbent polymer and at least one of synthetic fibers and regenerated fibers.

The absorbent material according to this invention will not lose its shape and does not require other members than the absorbent material itself to keep the shape of the absorbent material. This is due to the reason that the absorbent material is a woven or knitted fabric made of yarns and each of the yarns functions to restricts relative movement of the yarns.

Movement of the disposable diaper, the sanitary napkin or the like using the article of this invention is not restricted by the article because the absorbent material can be freely deformed to follow the deformation of the diaper or the napkin. In the diaper or the napkin using the absorbent material of this invention, body fluids can be reliably absorbed and contained by the absorbent material, so there is no anxiety that body fluids might flow back to the topsheet and stick to the wearer's skin.

### BRIEF DESCRIPTION OF THE DRAWINGS

FIG. 1 is a perspective view showing a disposable diaper as viewed from the side of a topsheet and partially cutaway;

FIG. 2 is a perspective view of an absorbent material;

FIG. 3 is a perspective view showing a yarn constituting the absorbent material;

FIG. 4 is an enlarged perspective view of a single synthetic fiber;

FIG. 5 is a perspective view showing another embodiment of the yarn;

FIG. 6 is a perspective view showing a sanitary napkin as viewed from the side of a topsheet and partially cutaway;

FIG. 7 is a perspective view of an absorbent material; and

FIG. 8 is a perspective view showing a yarn constituting the absorbent material

### DETAILED DESCRIPTION OF THE PREFERRED EMBODIMENTS

Details of a body fluid absorbent article according to this invention will be more fully understood from the description of an open-type disposable diaper a sanitary napkin using the article of this invention in reference to the accompanying drawings.

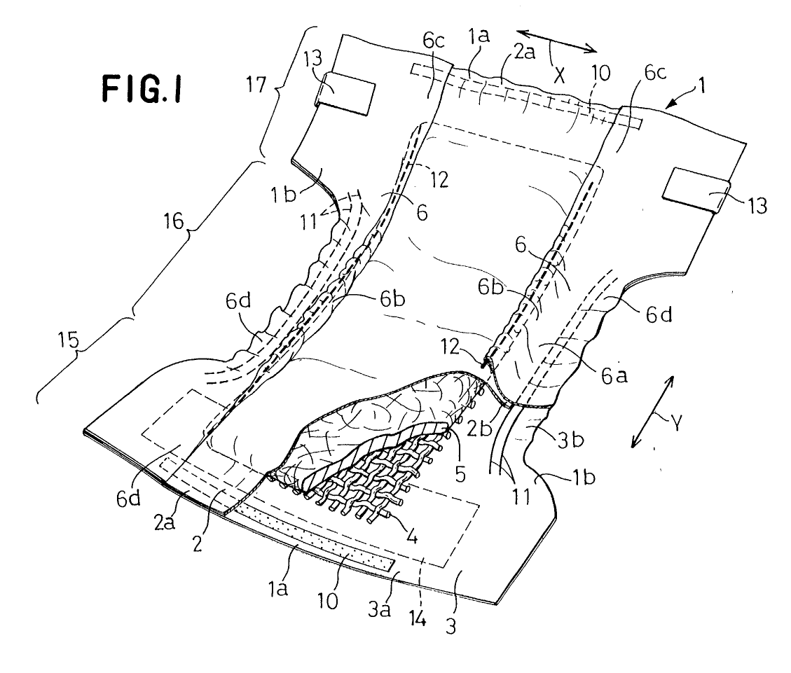

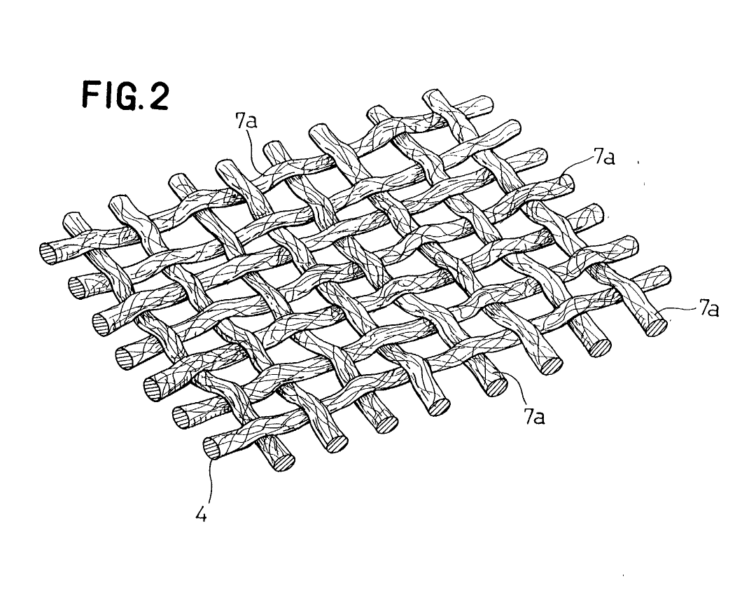

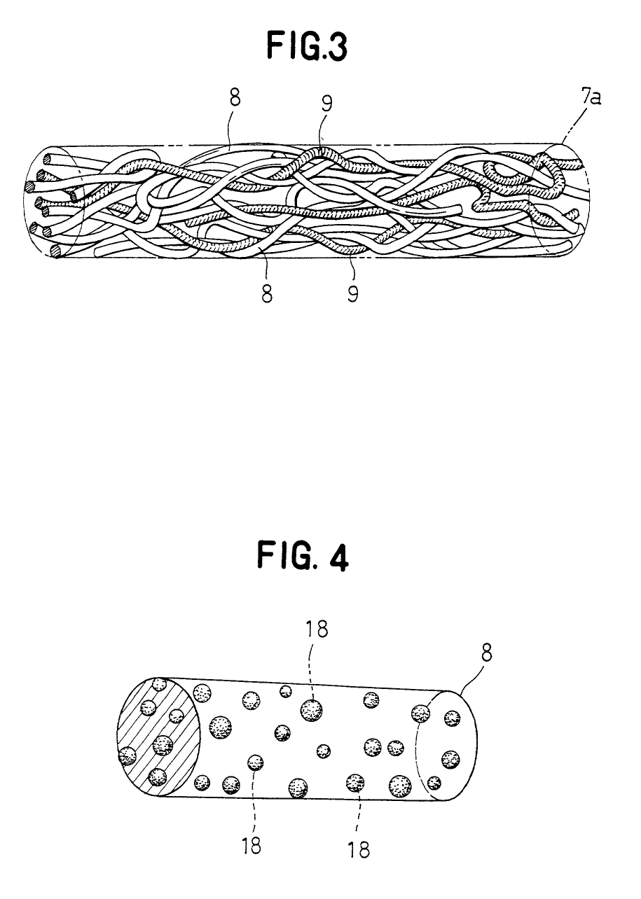

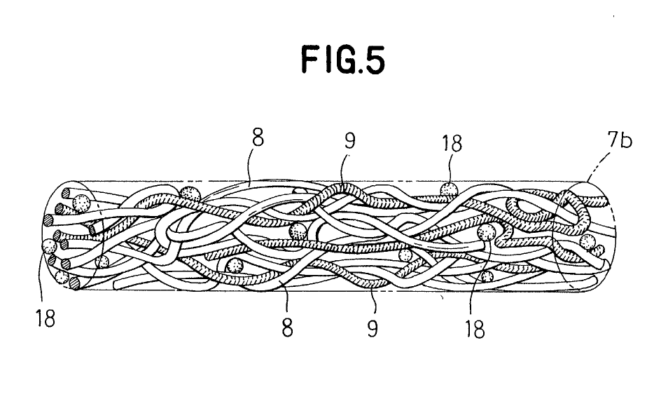

FIG. 1 is a perspective view showing a disposable diaper 1 as viewed from the side of a topsheet 2 and partially cutaway, FIG. 2 is a perspective view showing an absorbent material 4, FIG. 3 is a perspective view showing a yarn 7a constituting the absorbent material 4 and FIG. 4 is an enlarged perspective view of a synthetic fiber 8. FIG. 5 is a perspective view showing another embodiment of a yarn 7b. In FIG. 1, a transverse direction of the diaper 1 is indicated by an arrow X and its longitudinal direction is indicated by an arrow Y. An expression ‘inner surfaces’ of topsheet 2 and backsheet 3, respectively, used herein should be understood to be the surfaces facing the absorbent material 4 and an expression ‘outer surfaces’ of these sheets 2, 3, respectively, should be understood to be the surfaces facing away from the absorbent material 4.

The diaper 1 comprises the liquid-pervious topsheet 2 facing a wearer's skin, the liquid-impervious backsheet 3 facing away from the wearer's skin, and the absorbent material 4 and a fibrous assembly 5 both being disposed between the top- and backsheets 2, 3. The fibrous assembly 5 is placed upon the upper side of the absorbent material 4. The diaper 1 further comprises substantially liquid-impervious leak-barrier cuffs 6 lying on the outer side of the topsheet 2 and extending in the longitudinal direction.

The diaper 1 is composed, as viewed in the longitudinal direction, of a front waist region 15, a rear waist region 17 and a crotch region 16 extending between these two waist regions 15, 17 and contoured by longitudinally opposite ends 1a extending across the front and rear waist regions 15, 17 in the transverse direction and transversely opposite side edges 1b extending in the longitudinal direction. In the crotch region 16, the transversely opposite side edges 1b describe circular arcs which are convex inwardly in the transverse direction of the diaper 1.

The absorbent material 4 is joined to the inner surface of the backsheet 3. The fibrous assembly 5 is joined to the inner surface of the topsheet 2. The absorbent material 4 and the fibrous assembly 5 are joined together along the surfaces thereof opposed to each other.

The absorbent material 4 is a net-like woven fabric woven by the yarns 7a comprising synthetic fibers 8 and regenerated fibers 9. As will be apparent from FIG. 4, within the synthetic fibers 8, super-absorbent polymer particles 18 are included. Such synthetic fibers 8 may be obtained by spinning a molten thermoplastic synthetic resin blended with the super-absorbent polymer particles 18.

The synthetic fibers 8 are made of polyolefine-based fibers. As for the polyolefine-based fibers, at least one of polypropylene-based fibers and polyethylene-based fibers may be used. It is also possible to use core-sheath type conjugated fibers, side-by-side type conjugated fibers or sea-island type conjugated fibers of polyethylene/polypropylene. Alternatively, hollow fibers or fibers with an irregularly shaped cross-section may be used as the polyolefine-based fibers.

In addition to the polyolefine-based fibers, polyesteror polyamide-based fibers may be used as the synthetic fibers 8. It is also possible to blend at least two of the polyolefine-, polyester- and polyamide-based fibers as the synthetic fibers 8. As for the regenerated fibers 9, cellulose-based fibers such as rayon or acetate may be used.

The super-absorbent polymer particles 18 may be selected from a group of materials including a hydrolyzed compound of starch-acrylic acid graft copolymer, starch-based water absorbent resin obtained by neutralizing starch-acrylic acid graft copolymer, saponified vinyl acetate-acrylic ester copolymer, partially neutralized polyacrylic acid, and a polymer obtained from maleic anhydride-isobutylene copolymer and water-soluble ethylene-based unsaturated monomer. The super-absorbent polymer particles 18 may be incorporated either into both the synthetic fibers 8 and the regenerated fibers 9 or into the regenerated fibers 9 alone.

The fibrous assembly 5 is provided for the purpose of protecting the wearer from having an uncomfortable feeling when the wearer's skin is touched with the absorbent material 4 and serves also as a cushion. The fibrous assembly 5 is a mixture of the synthetic fibers, and the regenerated fibers and fluff pulp, which are compressed together to a desired thickness.

The yarn 7b shown in FIG. 5 comprises of the synthetic fibers 8 and the regenerated fibers 9 wherein the super-absorbent polymer particles 18 are adhered to these fibers 8, 9 around their outer surfaces by means of an adhesive (not shown). The adhesive may be selected from a group of materials including a hot melt adhesive and ethylene-glycidyl (metha)acrylate copolymer.

As for the yarn 7b shown in FIG. 5, it is also possible to utilize adhesive properties of the super-absorbent polymer particles 18 themselves to have these particles 18 deposited on the outer surfaces of these fibers 8, 9. In this case, the super-absorbent polymer particles 18 may be added with an adequate amount of water before having them deposited on the outer surfaces of the fibers 8, 9 followed by drying these super-absorbent polymer particles 18. It is enough for the yarn 7b of FIG. 5 to have the super-absorbent polymer particles 18 deposited onto at least one of the synthetic fibers 8 and the regenerated fibers 9.

Content of the super-absorbent polymer particles 18 in these yarns 7a, 7b is preferably in a range of 30-60 wt %. If the content of the super-absorbent polymer particles 18 is less than 30 wt %, a body fluid absorbing capacity of the absorbent material 4 will be decreased. With the yarn 7a wherein the synthetic fibers 8 contains within itself the super-absorbent polymer particles 18, the content of the super-absorbent polymer particles 18 exceeding 60 wt % would reduce the strength of the fibers 8 when the fibers 8 is wetted, so such anxiety is raised that the fibers 8 itself can not keep its shape. With the yarns 7b wherein the super-absorbent polymer particles 18 are adhered onto the outer surfaces of the synthetic fibers 8 as well as of the regenerated fibers 9, the content of the super-absorbent polymer particles 18 exceeding 60 wt % would make the super-absorbent polymer particles 18 more likely to be swollen by absorbing moisture, forming gel blocks. The super-absorbent polymer particles 18 with the gel blocks may prevent diffusion or absorption of body fluids in the absorbent material 4.

In the absorbent material 4, fineness of the synthetic fibers 8 and the regenerated fibers 9 is preferably in a range of 1.5-100 dtex. If the fineness of these fibers 8, 9 is less than 1.5 dtex, strength of these fibers 8, 9 would be decreased and easily damaged. If the fineness of these fibers 8, 9 exceeding 100 dtex, on the contrary, stiffness of the yarns 7a, 7b would be raised too high to keep desirable flexibility of the absorbent material 4.

The number of individual fibers 8, 9 in the yarns 7a, 7b, respectively, is preferably in a range of 20-800. If the number of the individual fibers 8, 9 is less than 20, the body fluid absorbing capacity of the absorbent material 4 would be decreased and flexural modulus as well as flexural recovering property would be also reduced. If the number of the individual fibers 8, 9 exceeds 800, diameter of the yarns 7a, 7b would be excessively increased and consequently thickness of the absorbent material 4 would be unacceptably increased.

Along the longitudinally opposite ends 1a, ribbon-like elastic members 10 associated with a waist-hole extending in the transverse direction and secured in a stretched state to the diaper 1. In the crotch region 16, the transversely opposite side edges 1b have elastic members 11 associated with leg-holes extending in the longitudinal direction, each of these elastic members 11 comprising a plurality of elastic elements and secured in a stretched state to the diaper 1.

The leak-barrier cuffs 6 extend across the crotch region 16 into the front and rear waist regions 15, 17 in the vicinity of the respective side edges 1b. Each of the cuffs 6 has a fixed side edge portion 6a extending across the crotch region 16 in the longitudinal direction, a free side edge portion 6b normally biased to rise on the topsheet 2 and longitudinally opposite fixed end portions 6c lying in the front and rear waist regions 15, 17, respectively, and collapsed inwardly in the transverse direction of the diaper 1. Of the cuffs 6, the fixed side edge portions 6a are joined to the transversely opposite side edges 1b, respectively, and the longitudinally opposite fixed end portions 6c are joined to the longitudinally opposite ends 1a, respectively.

Each of the cuffs 6 further includes a lateral portion 6d extending outwardly from the fixed side edge portion 6a in the transverse direction. The free side edge portion 6b has an elastic member 12 extending in the longitudinal direction and secured in a stretched state thereto. This elastic member 12 is wrapped with a part of the free side edge portion 6b.

As the diaper 1 is curved in the longitudinal direction with the topsheet 2 inside, contraction of the respective elastic members 12 causes the respective free side edges 6b of the cuffs 6 to be contracted and thereby to rise on the topsheet 2. In the diaper 1, the free side edge portions 6b of the cuffs 6 thus rising on the topsheet 2 form barriers against body fluids and prevent body fluids from leaking from the crotch region 16.

Of the diaper 1, the transversely opposite side edges 1b of the rear waist region 17 are provided with a pair of tape fasteners 13, respectively, and the backsheet 3 in the front waist region 15 is provided on its outer surface with a rectangular target tape strip 14 functioning as a landing zone for the tape fasteners 13.

Each of the tape fasteners 13 is made of a flexible plastic film and has a proximal end portion attached to the associated side edge 1b of the rear waist region and a free end portion adapted to be temporarily anchored on the target tape strip 14. Of the tape fastener 13, the free end portion is coated with self adhesive (not shown) so as to be temporarily fixed to the associated cuff 6 by means of adhesive. The target tape strip 14 is made of a flexible plastic film.

End portions 2a of the topsheet 2 as well as end portions 3a of the backsheet 3 extend outwardly beyond the opposite ends 1a of the absorbent material 4 in the longitudinal direction and these ends 2a, 3a are overlapped and joined to each other. The longitudinally opposite fixed end portions 6c of the respective cuffs 6 are joined to the respective end portions 2a of the topsheet 2. The elastic members 10 associated with the waist-hole are interposed between the end portions 2a of the topsheet 2 and the end portions 3a of the backsheet 3 and secured to these end portions 2a, 3a.

In the vicinity of the transversely opposite side edges 1b, the opposite side edge portions 2b of the topsheet 2 extend slightly outward of the opposite side edges of the absorbent material 4, and the opposite side edge portions 3b of the backsheet 3 and the lateral portions 6d of the respective cuffs 6 extend outwardly beyond the opposite side edge portions 2b of the topsheet 2. The side edge portions 2b and the side edge portions 3b overlap and are joined to each other, respectively. The side edge portions 3b and the lateral portions 6d overlap and are joined to each other, respectively. The fixed side edge portions 6a of the respective cuffs 6 are joined to the associated side edge portions 2b of the topsheet 2. The elastic members 11 associated with the leg-holes are interposed between the side edge portions 3b of the backsheet 3 and the lateral portions 6d of the cuffs 6 and secured to these side edge portions 3b and lateral portions 6d, respectively.

To wear the diaper 1, the opposite side edges 1b of the rear waist region 17 may be placed upon the opposite side edges 1b of the front waist region 15 and the free end portions of the tape fasteners 13 may be anchored on the target tape strip 14 by means of adhesive. With the front and rear waist regions 15, 17 connected to each other in this manner, the diaper 1 defines the waist-hole and the pair of leg-holes.

Of the diaper 1, the absorbent material 4 is provided in a form of woven fabric obtained from the yarns 7a, 7b, which serves to restrict relative movement of the yarns in the absorbent material 4 and thereby to prevent the shape of the absorbent material 4 from being lost. In other words, other members are not necessarily required to keep the shape of the absorbent material 4. Even when the diaper 1 is bent or twisted, the absorbent material 4 is freely deformed following such bending or twisting, so there is no anxiety that the absorbent material 4 might restrict movement of the diaper 1.

In the diaper 1, body fluids permeated through the topsheet 2 and the fibrous assembly 5 can be reliably absorbed and retained by the super-absorbent polymer particles 18 so that back flow of body fluids to the topsheet 2 as well as sticking of body fluids to the wearer's skin can be completely avoided.

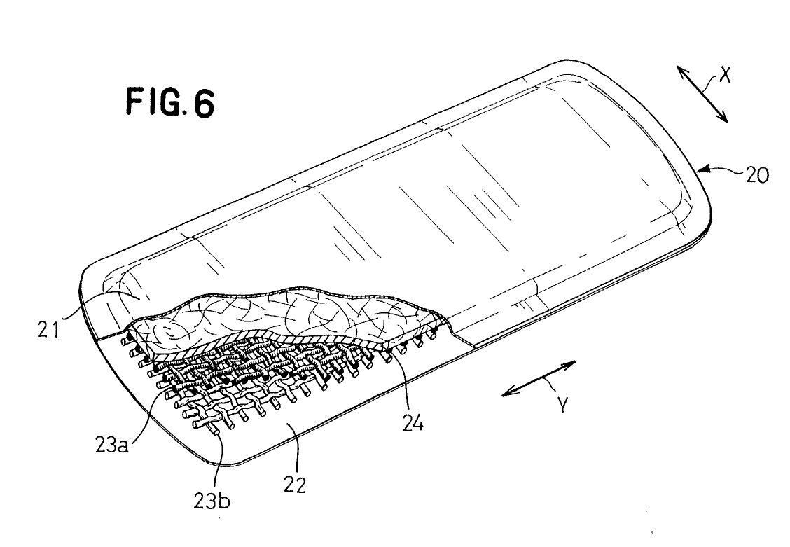

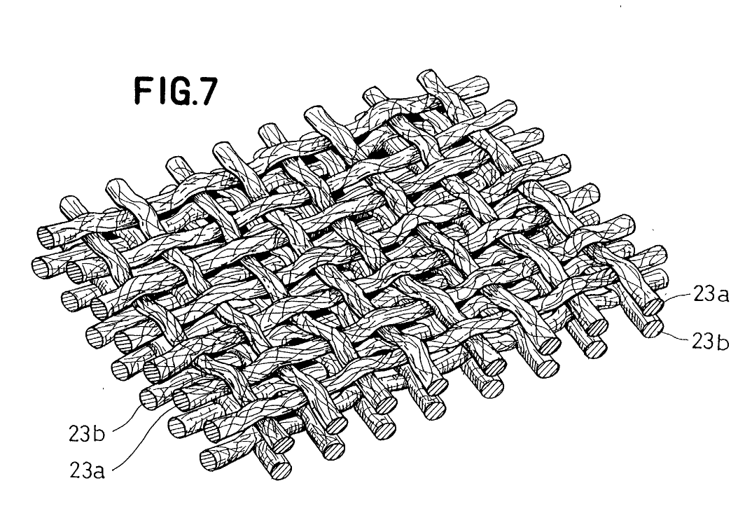

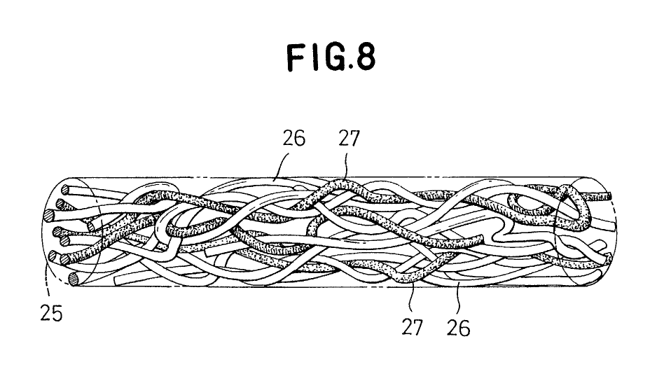

FIG. 6 is a perspective view showing a sanitary napkin 20 as viewed from the side of a topsheet 21 and partially cutaway, FIG. 7 is a perspective view showing absorbent materials 23a, 23b and FIG. 8 is a perspective view showing yarns 25 constituting the absorbent materials 23a, 23b. In FIG. 6, a transverse direction of the sanitary napkin 20 is indicated by an arrow X and its longitudinal direction is indicated by an arrow Y.

The napkin 20 presents a substantially rectangular shape which is longer in back-and-forth direction and comprises a liquid-pervious topsheet 21 facing a wearer's skin, a liquid-impervious backsheet 22 facing away from the wearer's skin, and the absorbent materials 23a, 23b and a fibrous assembly 24 both interposed between the top- and backsheets 21, 22. The fibrous assembly 24 lies on the upper surface of the absorbent materials 23a.

In the napkin 20, two absorbent materials 23a, 23b are layered one upon another in the thickness direction and joined together at mutually contacting points thereof. The absorbent material 23b is joined to the inner surface of the backsheet 22 and the fibrous assembly 24 is joined to the inner surface of the topsheet 21. The absorbent material 23a and the fibrous assembly 24 are joined together at mutually contacting points thereof.

In the napkin 20, the top- and backsheets 21, 22 extend outwardly beyond peripheral edges of these absorbent materials 23a, 23b and the fibrous assembly 24 in a circumferential direction and are joined to each other in the overlapped extensions of the top- and backsheets 21, 22. The outer surface of the backsheet 22 is coated with an adhesive (not shown) which adheres the napkin 20 temporarily to the inner side of a pair of shorts.

As will be apparent from FIG. 8, the absorbent materials 23a, 23b are a net-like woven fabric obtained by weaving yarns 25 comprising synthetic fibers 26 and super-absorbent fibers 27. The super-absorbent fibers 27 are obtained by spinning a super-absorbent polymer. More specifically, acrylic acid and an unsaturated vinyl monomer which can be formed to fibers are polymerized in aqueous solution, and its carboxylic acid is partially neutralized before subjected to dry spinning. Thereafter, a cross-linking reaction is undertaken to obtain the super-absorbent fibers 27, which is water insoluble. This technique is disclosed in Japanese Patent Publication No. 2613604.

The super-absorbent fibers 27 are not limited to that disclosed in the above-cited Japanese Patent Publication No. 2613604 and can be obtained also by a melt spinning ethylene-glycidyl (metha)acrylate copolymer. The term ‘(metha)acrylate’ used herein means acrylate and methacrylate.

Specific examples of the ethylene-glycidyl (metha)acrylate copolymer include those described below: ethylene-glycidyl acrylate copolymer, ethylene-glycidyl methacrylate copolymer, ethylene-glycidyl acrylate-glycidyl methacrylate copolymer, ethylene-glycidyl acrylate-vinyl acetate copolymer, ethylene-glycidyl acrylate-vinyl acetate-methyl acrylate copolymer, ethylene-glycidyl acrylate-methyl methacrylate copolymer, ethylene-glycidyl acrylate-vinyl acetate-methyl methacrylate copolymer, ethylene-glycidyl acrylate-ethylacrylate copolymer, ethylene-glycidyl acrylate-vinyl acetate-ethylacrylate copolymer, ethylene glycidyl methacrylate-vinyl acetate copolymer, ethylene-glycidyl methacrylate-methyl acrylate copolymer, ethylene-glycidyl methacrylate-vinyl-acetate-methyl acrylate copolymer, ethylene-glycidyl methacrylate-methyl acrylate copolymer, ethylene-glycidyl methacrylate-vinyl acetate-methyl methacrylate copolymer, ethylene-glycidyl methacrylate-vinyl acetate-ethyl acrylate copolymer, ethylene-glycidyl methacrylate-vinyl acetate-ethyl acrylate copolymer, ethylene-glycidyl acrylate-glycidyl methacrylate-vinyl acetate copolymer, and ethylene-glycidyl acrylate-glycidyl methacrylate-methyl acrylate copolymer.

In the absorbent material 23a, 23b, the content of the super-absorbent fibers 27 in the yarn 25 is preferably in a range of 30-60 wt % and fineness for the synthetic fibers 26 and the super-absorbent fibers 27 is preferably in a range of 1.5-100 dtex. If the content of the super-absorbent fibers 27 is less than 30 wt %, a body fluid absorbing capacity of the absorbent materials 23a, 23b would be decreased. If the content of the super-absorbent fibers 27 exceeds 50 wt %, on the contrary, strength of the super-absorbent fibers 27 would be decreased as the fibers 27 absorbs body fluids and is swollen. As a result, the super-absorbent fibers 27 would be separated from the synthetic fibers 26 and the yarns 25 constituting the absorbent materials 23a, 23b would be readily loosened. If the fineness of these fibers 26, 27 is less than 1.5 dtex, strength of these fibers 26, 27 would be decreased and particularly the super-absorbent fibers 27 would be readily damaged. On the contrary, if the fineness of these fibers 26, 27 exceeds 100 dtex, the yarns 25 would become too stiff and the desired flexibility of the absorbent materials 23a, 23b could not be expected.

The number of the fibers 26, 27 in each of the yarns 25, respectively, is preferably in a range of 20-800. If the number of the fibers 26, 27 in each of the yarns 25 is less than 20, a body fluid absorbing capacity of the absorbent materials 23a, 23b would be decreased and flexural modulus as well as flexural recovering property would be also reduced. If the number of the fibers 26, 27 exceeds 800, diameter of the yarns 25 would be extremely enlarged and consequently thickness of the absorbent materials 23a, 23b would be unacceptably increased.

Alternatively, these absorbent materials 23a, 23b may be formed by yarns composed of regenerated fibers and super-absorbent fibers, or by yarns composed of synthetic fibers, regenerated fibers and super-absorbent fibers. The synthetic fibers, the fibrous assembly and the regenerated fibers may be similar to those in FIG. 1.

In the napkin 20, body fluids having permeated through the topsheet 21 and the fibrous assembly 24 can be reliably absorbed and retained by the super-absorbent fibers 27 forming the absorbent materials 23a, 23b so that back flow of body fluids to the topsheet 21 as well as sticking of body fluids to the wearer's skin can be completely avoided.

While these absorbent materials 4, 23a, 23b have been illustrated and described as a net-like woven fabric having apertures between the adjacent yarns 7a, 7b, 25, it should be understood that they are not limited to the net-like woven fabric. They can be provided in a form of fabric having substantially invisible apertures or gaps between the adjacent yarns 7a, 7b, 25. These absorbent materials 4, 23a, 23b are not limited to a woven fabric and may be knitted ones made of the yarns.

While the diaper 1 as well as the napkin 20 have been illustrated and described as having the fibrous assemblies 5, 24 placed on the upper surfaces of the absorbent materials 4, 23a, 23b, the fibrous assemblies 5, 24 are not essential and it is also possible to interpose nothing but the absorbent materials 4, 23a, 23b between the topsheets 2, 21 and the backsheets 3, 22. Furthermore, an arrangement is also possible in which the diaper 1 or the napkin 20 is provided with more than two absorbent materials stacked in the thickness direction.

The topsheets 2, 21 may be formed of a hydrophilic fibrous nonwoven fabric or a plastic film with a large number of micro-pores. The backsheets 3, 22 may be formed of a hydrophobic fibrous nonwoven fabric, a liquid-impervious plastic film, two-layers of hydrophobic fibrous nonwoven fabric laminated with each other or a composite sheet composed of a hydrophobic fibrous nonwoven fabric and a plastic film joined to this hydrophobic fibrous nonwoven fabric. The leak-barrier cuffs 6 may be formed of a hydrophobic fibrous nonwoven fabric.

The backsheet 3, 22 and the leak-barrier cuffs 6 may be also formed of a composite nonwoven fabric consisting of a highly water-resistant fibrous nonwoven fabric by melt blown method sandwitched by and two-layers of fibrous nonwoven fabric having high strength and flexibility by spun bond method.

Nonwoven fabric used herein may be selected from a group of fabrics including those obtained by spun lace-, needle punch-, melt blown-, thermal bond-, spun bond-, chemical bond- and air through-processes. Component fibers of such nonwoven fabric may be selected from a group of materials consisting of polyolefine-, polyester- and polyamide-based fibers and core-sheath type or side-by-side type conjugated fibers of polyethylene/polypropylene or polyethylene/polyester.

Joining of the top- and backsheets 2, 21, 3, 22 and the leak-barrier cuffs 6 as well as of the absorbent materials 4, 23a, 23b and the fibrous assemblies 5, 24, and attachment of the elastic members 10, 11, 12 may be carried out using a hot melt adhesive or welding technique such as heat-sealing or ultrasonic sealing.

These absorbent materials 4, 23a, 23b are applicable not only to the open-type diaper 1 and the sanitary napkin 20 but also to a pants-type diaper with its front and rear waist regions previously connected to each other and a liquid-absorbent pad adapted to be attached to a diaper cover in actual use. In addition, the absorbent materials are applicable also to a blood stanching bandage, a sweat absorbent sheet and a mother's milk pad.

What is claimed is:

1. A body fluid absorbent article comprising:
an absorbent material comprising at least one of a woven fabric and a knitted fabric made of yarns which are composed of super-absorbent polymer particles and at least one of synthetic fibers and regenerated fibers which are united with said super-absorbent polymer particles.

2. The article according to claim 1, wherein said super-absorbent polymer particles are blended with a material of said at least one of said synthetic fibers and said regenerated fibers.

3. The article according to claim 1, wherein said super-absorbent polymer particles are adhered to an outer surface of said at least one of said synthetic fibers and said regenerated fibers by an adhesive.

4. The article according to claim 1, wherein said super-absorbent polymer particles are adhered on an outer surface of said at least one of said synthetic fibers and said regenerated fibers by adhesive properties.

5. The article according to claim 1, wherein a content of said super-absorbent polymer particles in said yarns is in a range of 30-60 wt %.

6. The article according to claim 1, wherein said synthetic fibers and said generated fibers have a fineness in a range of 1.5-100 dtex and the number of said synthetic fibers and said regenerated fibers in each of said yarns is in a range of 20-800.

7. The article according to claim 1, wherein said synthetic fibers are made of at least one of polyolefine-based fibers, polyester-based fibers, and polyamide-based fibers, and said regenerated fibers are made of cellulose-based fibers.

8. The article according to claim 1, wherein said absorbent material is interposed a liquid-pervious topsheet facing a wearer's skin and a liquid-impervious backsheet facing away from the wearer's skin.

9. The article according to claim 1, wherein at least double layers of said absorbent material are stacked in a thickness direction of said absorbent material.

10. A body fluid absorbent article comprising:
an absorbent material comprising at least one of a woven fabric and a knitted fabric made of yarns which are composed of super-absorbent fibers obtained by spinning a super-absorbent polymer and at least one of synthetic fibers and regenerated fibers.

11. The article according to claim 10, wherein a content of said super-absorbent fibers in said yarns is in a range of 30-50 wt % and a fineness of said super-absorbent fibers is in a range of 1.5-100 dtex.

12. The article according to claim 10, wherein said synthetic fibers and said generated fibers have a fineness in a range of 1.5-100 dtex and the number of said synthetic fibers and said regenerated fibers in each of said yarns is in a range of 20-800.

13. The article according to claim 10, wherein said synthetic fibers are made of at least one of polyolefine-based fibers, polyester-based fibers and polyamide-based fibers, and said regenerated fibers are made of cellulose-based fibers.

14. The article according to claim 10, wherein said absorbent material is interposed a liquid-pervious topsheet facing wearer's skin and a liquid-impervious backsheet facing away from the wearer's skin.

15. The article according to claim 10, wherein at least double layers of said absorbent material are stacked in a thickness direction of said absorbent material.
